(19) United States
(12) Patent Application Publication
(10) Pub. No.: US 2013/0012719 A1
(43) Pub. Date: Jan. 10, 2013

(54) Convenient Synthesis of Azolines to Azoles

(75) Inventors: Travis J. Williams, Los Angeles, CA (US); Anna C. Dawsey, Los Angeles, CA (US); Vincent Li, Los Angeles, CA (US); Kimberly C. Hamilton, Edison, NJ (US); Jianmei Wang, Riverside, CA (US)

(73) Assignee: University of Southern California, Los Angeles, CA (US)

(21) Appl. No.: 13/543,403

(22) Filed: Jul. 6, 2012

Related U.S. Application Data
(60) Provisional application No. 61/505,752, filed on Jul. 8, 2011.

(51) Int. Cl. C07D 277/10; C07D 487/00; C07D 263/14

(52) U.S. Cl. 548/181; 548/201; 548/239; 548/237

(57) ABSTRACT

Azolines are oxidized in the presence of a copper-containing catalyst to azoles in the presence of molecular oxygen. A synthetic scheme converting azolines azoles is also provided.

### A CROSS-REFERENCE TO RELATED APPLICATIONS

This application claims the benefit of U.S. provisional Application No. 61/505,752 filed Jul. 8, 2011, the entire disclosure of which is incorporated herein by reference.

### STATEMENT REGARDING FEDERALLY SPONSORED RESEARCH OR DEVELOPMENT

The invention was made with American Cancer Society support under Contract No. IRG-58-007-48. The American Cancer Society has certain rights to the invention.

### TECHNICAL FIELD

In at least one aspect, the present invention is related to methods for synthesizing azoles from azolines.

### BACKGROUND

Azoles are ubiquitous structural components in biologically active natural products (FIG. 1) with important medicinal properties that include anticancer, anti-inflammatory, antiviral, antifungal, and antibiotic activity. Thiazole-containing antibiotic agents include myxothiazol and the thiopeptide antibiotics (micrococcin, thiostrepton, amythiamicin D, promothiocin A, and nocathiacin 1, among others). These sulfur-containing heterocycles are also found embedded in natural products that exhibit anticancer activity such as mechercharmycin A, patupilone (epothilone B), riboxamide (tiazofurin), and synthetic chemotherapeutic candidates such as ATCAA, a thiazole-containing compound that exhibits cytotoxic behavior towards prostate cancer and melanoma.

Although azoles are prevalent throughout medicinal and natural products chemistry, we know of no catalytic conditions for azoline oxidation. Various conditions, which involve either a toxic waste stream or a stoichiometric amount of a metal reagent, effect thiazoline oxidation. Such reagents include K₃Fe(CN)₆, Hg(OAc)₂, NiO₂, Cu^{I}/Cu^{II}, BrCC₁₃, and MnO₂. In each of these cases the stoichiometric waste stream introduces disposal cost and environmental impact when these reactions are practiced at production scale. Further, as this work was in progress, aerobic conditions for thiazoline oxidation based on K₂CO₃/DMF solutions have appeared. These are efficient for aerobic oxidation of many electron poor azolines.

Accordingly, there is a need for improved synthetic methods for forming azole compounds.

### SUMMARY OF THE INVENTION

Against this prior art background, a method of forming an azole is provided. The method comprises:
a) reacting a compound having formula (2) with a copper-containing catalyst in the presence of a base or proton acceptor to form a compound having formula (2):

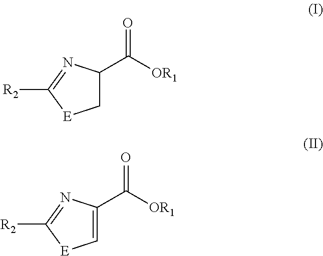

wherein:

R₁ is C₁-C₁₀ alkyl;

R₂ is an optionally substituted phenyl, optionally substituted aryl, or optionally substituted heteroaryl; and

E is O, S, or N.

In another embodiment, a second method of forming an azole without using a copper catalyst is provided. The method comprises:
a) reacting a compound having formula (I) with a base or proton acceptor in the presence of molecular oxygen to form a compound having formula (II):

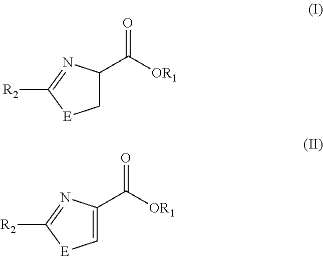

wherein:

R₁ is C₁-C₁₀ alkyl;

R₂ is an optionally substituted phenyl, optionally substituted aryl, or optionally substituted heteroaryl; and

E is O, S, or N.

### BRIEF DESCRIPTION OF THE DRAWINGS

Exemplary embodiments of the present invention will become more fully understood from the detailed description and the accompanying drawings, wherein:

FIG. 1 provides examples of thiazole containing bioactive compounds;

FIG. 2 provides Scheme 1 showing catalytic aerobic oxidation of thiazolines;

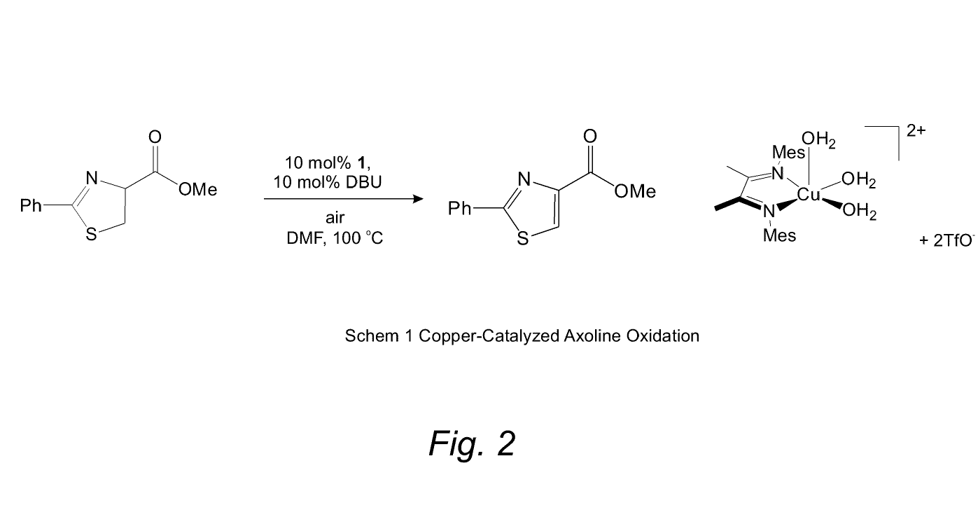

FIG. 3 provides Scheme 2 showing the synthesis and molecular structure of Complex 1. Ellipsoids are drawn at the 50% probability level. Selected bond distances (Å): Cu—N1=1.99; Cu—N2=2.00; Cu—O1=1.99; Cu—O2=1.97; Cu—O3=2.21. The largest spheroids represent peaks in the difference map, which are likely results of O—H bonds;

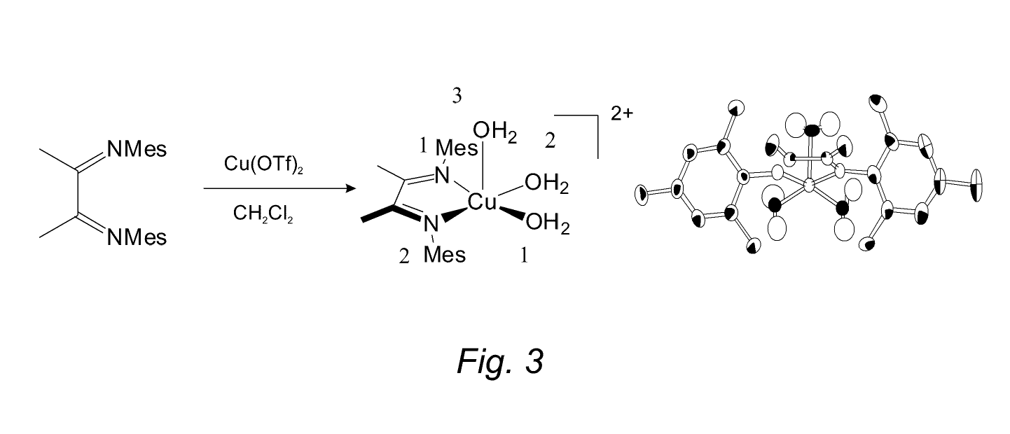

FIG. 4 provides Table 1 which contains information regarding the optimization of Cu^{II}-catalyzed oxidation conditions;

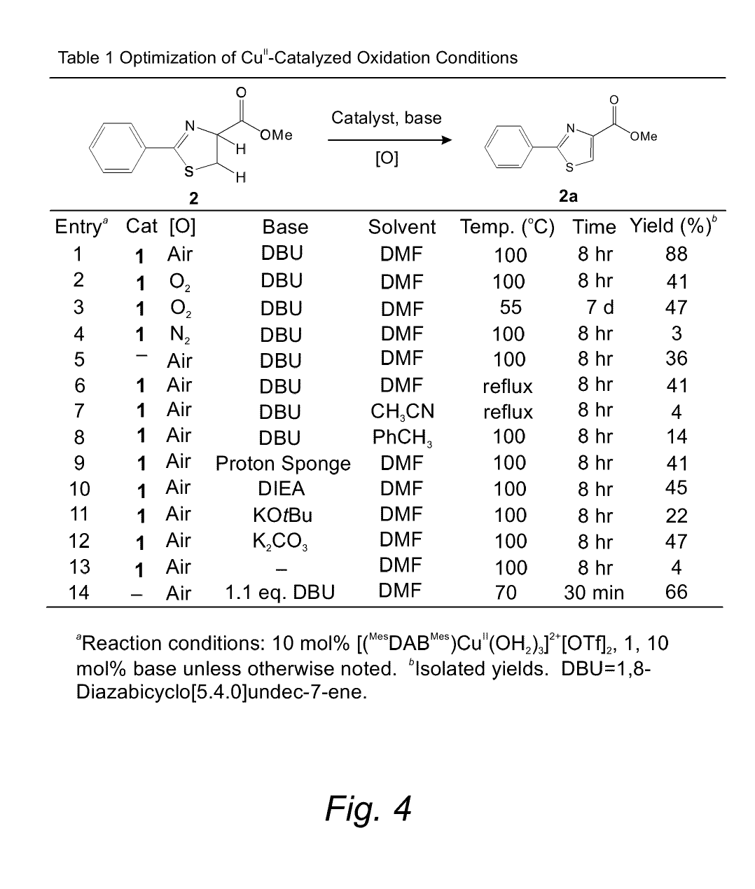

FIG. 5 provides Table 2 which contains information regarding the ligand screen of copper catalyzed oxidation of thiazoline 2 to thiazole 2a;

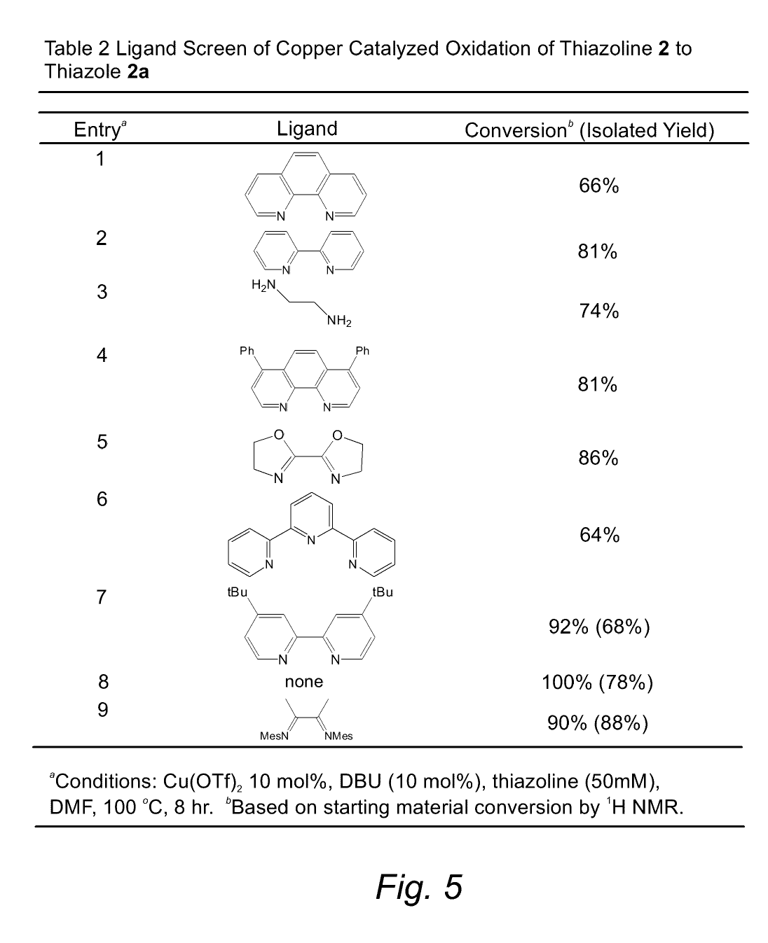

FIG. 6 provides Table 3 which contains information regarding the scope of Cu^{II} catalyzed oxidation conditions;

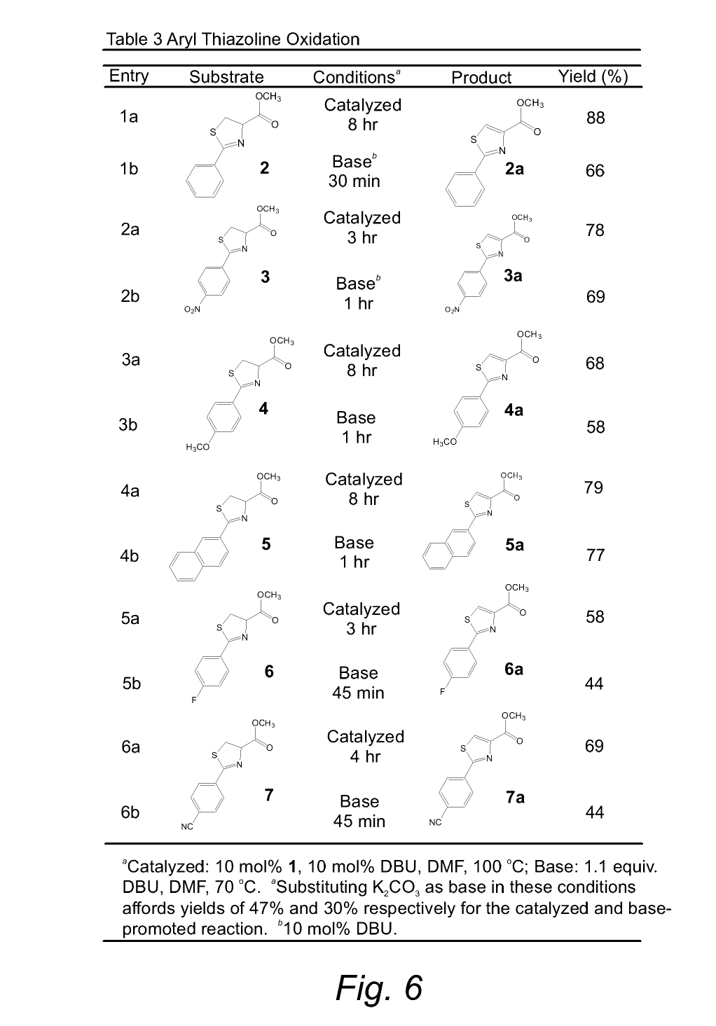

FIG. 7 provides Table 4 which contains information regarding the scope of Cu^{II} catalyzed oxidation conditions;

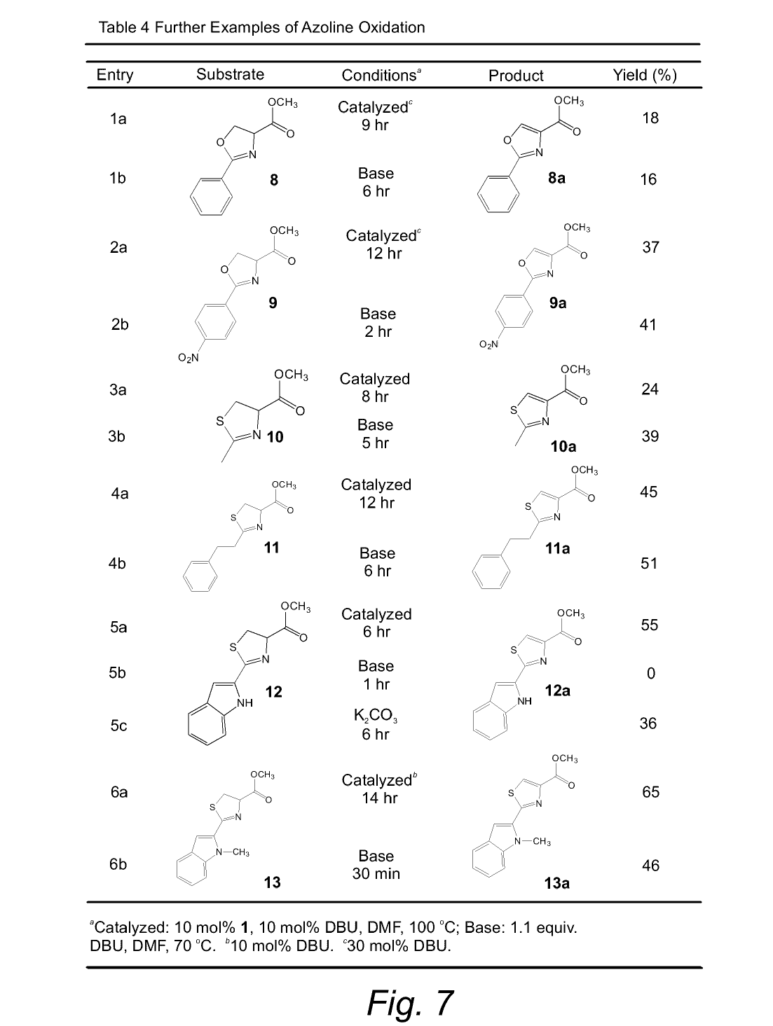

FIG. 8 provides Table 5 which contains information regarding scalability;

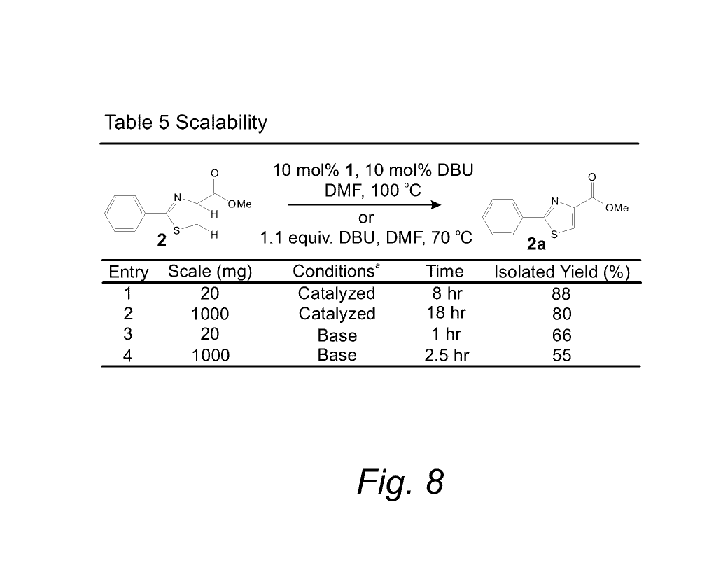

FIG. 9 provides Scheme 3 showing a potential oxidation mechanism for some embodiments of the invention; and

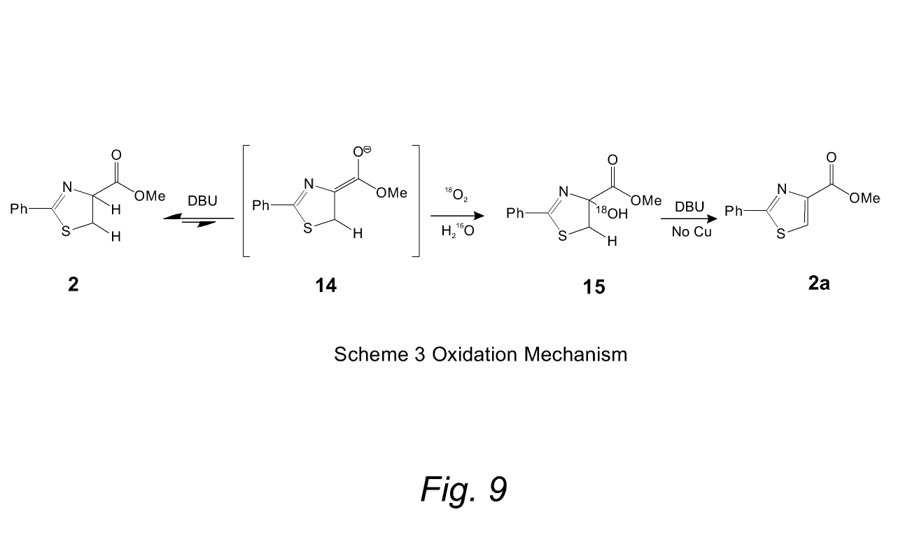

FIG. 10 provides intermediates in potassium carbonate-mediated oxidation.

### DETAILED DESCRIPTION OF EXEMPLARY EMBODIMENTS

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

Abbreviations:

TfO- or -OTf stands for Trifluoromethanesulfonate;

DMF stands for dimethylformamide;

DCM stands for dichloromethane;

DBU=1,8-Diazabicyclo[5.4.0]undec-7-ene; and

^{Mes}DAB^{Mes} stands for

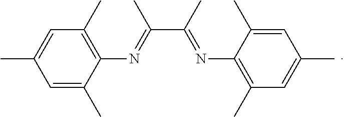

In an embodiment, a method of forming an azole is provided. The method comprises:
a) reacting a compound having formula (I) with a copper-containing catalyst in the presence of a base or proton acceptor to form a compound having formula (II):

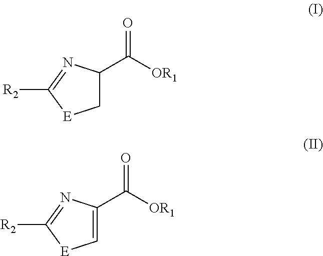

wherein:

R₁ is C₁-C₁₀ alkyl;

R₂ is an optionally substituted phenyl, optionally substituted C₅-C₁₈ aryl, or optionally substituted C₅-C₁₈ heteroaryl; and

E is O, S, or N.

In a refinement, R₁ is methyl, ethyl, butyl or pentyl and E is O or S.

The present embodiment, as set forth in Scheme 1, includes catalytic copper-based conditions for aerobic azoline oxidation which improves the scope of aerobic oxidation conditions to include electron donating substituents and scalability of the reaction while minimizing metallic waste stream. advantageously, these conditions are low cost. For example compound 2a of scheme 1 is commercially available for $22,500 g⁻¹ but can be prepared in route of the present embodiment for <$28 g⁻¹.

In a refinement of the embodiments set forth above, the copper-containing catalyst has formula (III):

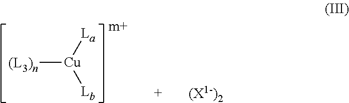

wherein:

Lₐ, L_{b}, and L₃ are each independently two electron ligands;

X¹⁻ is a negatively charged counter ion;

Cu is in Cu(I) or Cu(II)

n is 0, 1, 2, or 3; and

m is 0, 1, or 2.

Examples of negatively charge counter ions include, but are not limited to halide (e.g., Cl⁻, Br⁻, I⁻, etc), CF₃SO₃⁻, C₁₋₅ alkoxide, C₁₋₅ carboxylate, and the like. It should be appreciated that Lₐ, L_{b}, and L₃ can be a two electron ligand, a multidentate ligand (e.g., a bidentate ligand), charged ligand (e.g., −1 charged), a neutral ligand, and combinations thereof. Examples of Lₐ and L_{b} include, but are not limited to, H₂O, NH₃, C₁₋₅ primary amines, C₂₋₆ secondary amines, C₃₋₉ tertiray amines, PH₃, C₁₋₅ primary phosphines, C₂₋₆ secondary phosphine, C₃₋₉ tertiary phosphines, C₁₋₅ alcohols, CO, N₂, C₂₋₈ alkenes, C₂₋₈ alkynes, and the like. In a refinement, L₃ is a neutral ligand. Examples of neutral ligands for L₃ include, but are not limited to, H₂O, NH₃, C₁₋₅ primary amine, C₂₋₆ secondary amines, C₃₋₉ tertiary amines, PH₃, C₁₋₅ primary phosphine, C₂₋₆ secondary phosphines, C₃₋₉ tertiary phosphines, C₁₋₅ alcohols, CO, N₂, C₂₋₈ alkenes, C₂₋₈ alkynes, and the like. In another embodiment, L₃ is a negatively charged ligand. Examples of negatively charged ligands for L₃ include, but are not limited to, CF₃SO₃⁻, C₁₋₅ alkoxide, C₁₋₅ carboxylate, and the like.

In another refinement, the copper-containing catalyst has formula (IV):

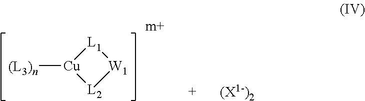

wherein:

L₁ and L₂ are dentates in a bidentate ligand L₁W₁L₂;

L₃ is a neutral ligand;

n is from 0, 1, 2, or 3;

W₁ is an absent or a C₁₋₁₈ hydrocarbon moiety attached to L₁ and L₂

X¹⁻ is a negatively charged counter ion;

Cu is in Cu(I) or Cu(II)

n is 0, 1, 2, or 3; and

m is 0, 1, or 2.

X¹⁻ is a negatively charged counter ion.

Examples of negatively charge counter ions include, but are not limited to halide (e.g., Cl⁻, Br⁻, I⁻, etc), CF₃SO₃⁻, C₁₋₅ alkoxide, C₁₋₅ carboxylate, and the like. In a refinement, L₃ is a neutral ligand. Examples of neutral ligands for L₃ include, but are not limited to, H₂O, NH₃, C₁₋₅ primary amines, C₂₋₆ secondary amines, C₃₋₉ tertiray amines, PH₃, C₁₋ₛ primary phosphines, C₂₋₆ secondary phosphines, C₃₋₉ tertiray phosphines, C₁₋₅ alcohols, CO, N₂, C₂₋₈ alkenes, C₂₋₈ alkynes, and the like. In another embodiment, L₃ is a negatively charged ligand. Examples of negatively charged ligands for L₃ include, but are not limited to, CF₃SO₃⁻, C₁₋₅ alkoxide, C₁₋₅ carboxylate, and the like. Table 2 provides examples for bidentate ligand L₁W₁L₂.

In another embodiment, a second method of forming an azole which does not use a copper-containing catalyst is provided. The method comprises:
a) reacting a compound having formula (I) with a base or proton acceptor in the present of molecular oxygen to form a compound having formula (II):

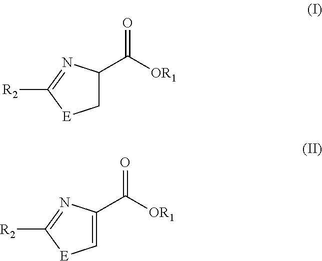

wherein:

R₁ is C₁-C₁₀ alkyl;

R₂ is an optionally substituted phenyl, optionally substituted aryl, or optionally substituted heteroaryl; and

E is O, S, or N.

In a refinement, R₂ is methyl, ethyl, butyl or pentyl. In another refinement, E is S or N.

In the embodiments set forth above, the compound having formula (I) is selected from the group consisting of optionally compounds having formula (V) and (VI):

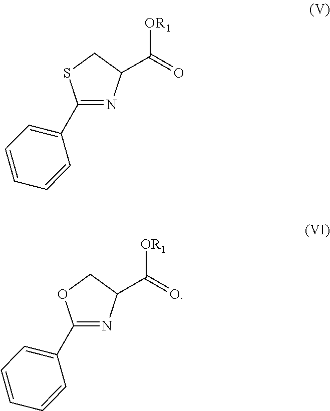

In a refinement, the phenyl group in compounds (V) or (VI) is substituted with C₁₋₆ alkyl, fluorine, chlorine, bromine, cyano, or nitro.

Examples of compound (I) are selected from the group consisting of:

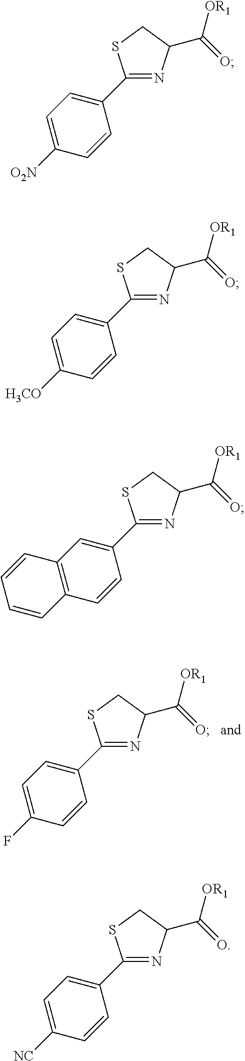

Additional examples of compound (I) is selected from the group consisting of:

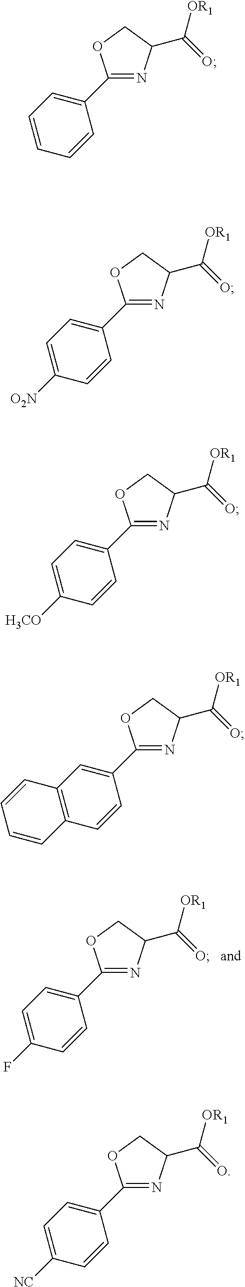

In other variation, R₂ in the compounds having formula (I) and (II) are:

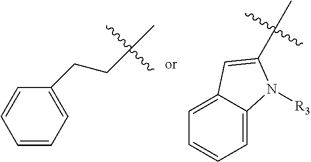

and R₃ is hydrogen or C₁₋₁₀ alkyl.
In the embodiments set forth above. Table 3 provides additional examples for compounds having formula (I) (substrates) and formula (II) (products).

In still another refinement of the embodiments set forth above, the reaction of step a) is performed in the presence of molecular oxygen.

In yet another refinement of the embodiments set forth above, the base or proton acceptor is 1,8-diazabicyclo[5.4.0]undec-7-ene, 1,8-Bis(dimethylamino)naphthalene (proton Sponge™), 1,8-bis(hexamethyltriaminophosphazenyl)naphthalene, diisopropyl ethyl amine, potassium tert-butoxide, or potassium carbonate.

The following examples illustrate the various embodiments of the present invention. Those skilled in the art will recognize many variations that are within the spirit of the present invention and scope of the claims.

### Results and Discussion

### Synthesis and Characterization of Copper Complexes

Copper complex 1 is prepared in two steps without need for chromatography from 2,3-butanedione and the corresponding trimethylaniline with the intermediacy a known diazabutadiene ligand, [^{Mes}DAB^{Me}] (Scheme 2). The structure of 1 is assigned by single-crystal X-ray diffraction. In this case copper adopts a distorted square pyramidyl geometry in which copper(II) appears to be a 19-electron metal center. The analogous (4,7-diphenylphenanthroline)-ligated complex 1a has a similar structure.

### Oxidation Reactions of Azolines

### Optimization of Reaction Conditions

Table 1 illustrates the optimization of catalytic aerobic oxidation conditions for the transformation of thiazoline 2 to thiazole 2a. Comparable results were observed upon screening other bidentate ligands for copper (vide infra), however, optimization and scope studies were performed solely with catalyst 1. Table 1 summarizes the optimization studies. Entries 1-4 demonstrate that although O₂ is essential for the reaction (entry 4), air is a more effective oxygen source than 1 atmosphere of O₂ (compare entries 1 and 2). Repeating the O₂ experiment (entry 2) at 55° C. did not improve this reaction (entry 3).

The copper-free background reaction (Table 1, entry 5) has an appreciable rate and results in product formation in 36% yield. Along these lines, entry 14 illustrates that in the presence of 1.1 molar equivalents DBU, oxidation reaches 66% yield (>99% conversion) in only 30 minutes. Solvents screening include DMF, DCM, CH₃CN, and PhCH₃ (entries 6-8); none was superior to the original DMF conditions. Neither Hiinig's base (entry 10) nor t-butoxide (entry 11) is as effective as DBU in these conditions, but both are superior to base-free conditions (entry 13). This result highlights the relative utility of catalytic and base-promoted conditions with an electon-neutral substrate. Importantly, it is observed that in a direct comparison with thiazoline (2), DBU conditions compare favorably to analogous K₂CO₃ conditions (compare entries 1 and 12).

Table 2 shows that the ligand used on copper has little influence in the outcome of the conversion of 2 to 2a. We found comparable results upon screening several nitrogen-based ligands for copper (entries 1-7). Among these, the diimine system found in 1 (entry 9) and ligand-free conditions (entry 8) afforded the best conversions, with the former affording a superior isolated yield.

Conditions were tested against a variety of thiazoline substrates (Table 3). Substrates with aryl substituents in the 2-position demonstrated good yields with a range of electron withdrawing and electron donating groups in the para-position. Electron-withdrawing groups such as aryl fluoride and nitrile (entries 5a, 6a) do not impede oxidation; more importantly, an electron-rich thiazoline is tolerated (entry 3a). A sensitive substrate and excellent synthetic handle such as the p-cyano thiazoline (7) shows a significant advantage in yields 69% vs. 9% when using the catalytic method of the invention versus aerobic K₂CO₃.Error! Bookmark not defined. Further, yields of 88% and 66% with DBU as base in the respective presence and absence of copper are an interesting contrast to yields 47% and 30% for otherwise identical reactions run with K₂CO₃ (1 equiv.) as base.

### Oxazolines

Oxazolines (Table 4, entries 1 and 2) were tested against the catalytic conditions with less success. Yields of the corresponding oxazoles are lower than those of the thiazole series. The reason for this difference is not clear, but it is suspected that the presence of a more polarizable sulfur center in an intermediate enolate (14, Scheme 3, vide infra) facilitates oxygen transfer. Evidence of an S-oxidation pathway is not observed, although such a mechanism cannot be eliminated.

Similarly, thiazolines containing 2-alkyl substituents proved difficult to oxidize and afforded lower yields than the 2-aryl thiazole counterparts (entries 3 and 4).

### Copper-Free, Base-Mediated Oxidation

Many of these reactions produce reasonable yields in the presence of base alone (e.g. Table 1, entry 14). Reactions run in the absence of copper with stoichiometric base generally have lower, but comparable yields to their catalytic counterparts but with advantageous, reduced reaction times. Yields for base promoted reactions are summarized in Tables 3 and 4 alongside the results for catalytic oxidation. It is important to note that these base-promoted reactions are apparently faster because they involve a molar excess of base whereas catalytic conditions involve only 10 mol percent each of copper and DBU.

The base conditions demonstrate increased yields in both the 2-substituted alkyl substrates (Table 4, entries 3b and 4b) and oxazoline containing substrate (entry 2b), while the catalytic conditions appear higher yielding in other cases. Particularly in situations of more electron rich thiazolines, catalytic conditions provide increased yields. It is suspected that the advantage in yield for the catalytic conditions is related to the minimization of intermolecular side reactions.

When a thaizoline substrate with a 2-substituted heterocycle, e.g. indole (entry 5), is subjected to DBU conditions, no product formation is observed. However, successful oxidation in 55% yield is achieved by application of catalytic conditions. When Yao et al.'s conditionsError! Bookmark not defined. were applied to the indole substrate (12, table 4, entry 5c) a yield of 36% was obtained. An N-methylindole-bearing substrate (13, entry 6) was subsequently subjected to both catalytic and base conditions, which lead to good yields in each case. These data show that in the presence of labile protons, as in indole, our catalyst proves superior for thiazoline oxidation.

### Scalability

The catalytic conditions are advantageous when the reaction is run on larger scale (Table 5), which is important if this transformation is to be used for material throughput. Thiazoline 2 is successfully oxidized on a 1 g scale to afford 80% yield of the thiazole 2a when copper conditions are utilized (entry 2). The base-mediated reaction is less efficient at this scale (entry 4).

### Mechanism

### Intermediates

We have made some observations that help us understand the reaction intermediates (scheme 3). We propose initial enolization of 2 followed by installation of an angular hydroxide (15). Notably, isolation and characterization of 15 confirms its presence in the reaction under copper-free conditions; independent conversion of 15 to 2a in the presence of DBU, with or without copper, provides evidence of its kinetic competence. Thus, it is believed that 2 is enolized to form a intermediate 14, which is oxidized either by a copper oxo species or dioxygen itself to give angular hydroxide 15.

We report that the angular hydroxide comes from O₂ as opposed to H₂O because we observe no incorporation of ¹⁸O when the reaction is run in the presence of H₂¹⁸O (see Supporting Information). Along these lines, the presence of a radical inhibitor (BHT, butylated hydroxytoluene, or tocopherol, vitamin E) does not affect the efficiency of the copper-catalyzed or stoichiometric base-promoted oxidation of 2. Therefore, a long-lived radical intermediate in either reaction is suspected. Further, addition of water does not provide increased yield or rate in either copper-catalyzed or stoichiometric base-promoted oxidation of 2.

### Intermediate Putative Peroxide

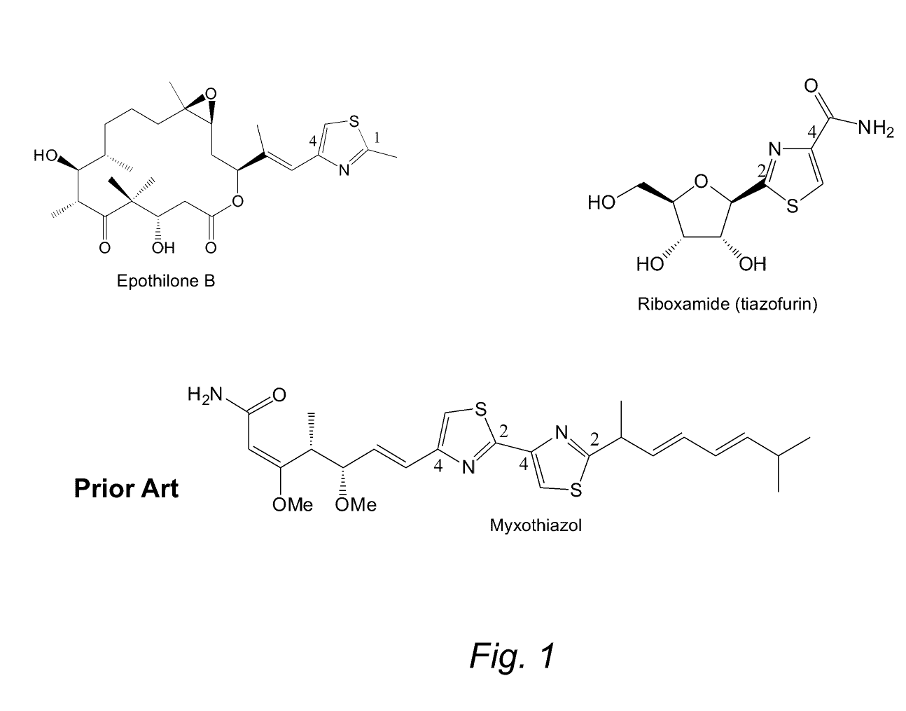

The conditions of the present examples do not involve the intermediacy of a long-lived hydroperoxide species. Yao et. al. report that under potassium carbonate conditions, a long-lived tertiary peroxide intermediate intervenes 14 and 15 in the oxidation mechanism as characterized by TLC evidence. By contrast, intermediate species in the reaction mixture other than 2, 15, and 2a are not observed when the oxidation is run with either our copper catalyzed conditions or stoichiometric DBU. By contrast, a species consistent with the putative peroxide is observed when the reaction is run under potassium carbonate conditions. This is illustrated in FIG. 1.

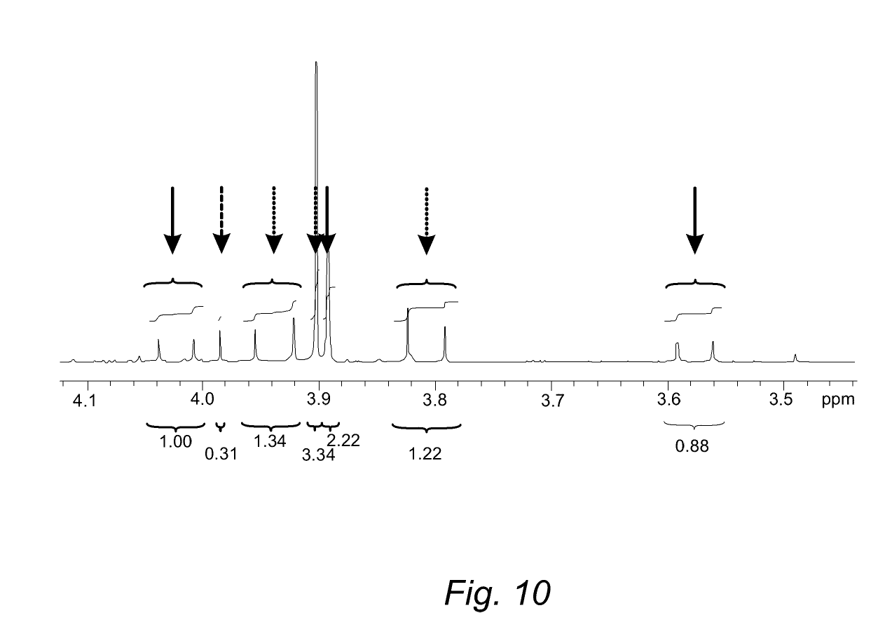

FIG. 10 illustrates the intermediate species that are observed in potassium carbonate-mediated oxidation. The thiazole product is evident from its methyl ester peak, highlighted with a dashed line. Each of 15 (solid line) and the putative peroxide intermediate (dotted line) are represented both by their methyl esters at 3.9 ppm and by a pair of doublets corresponding to their C5 methylene protons. The low concentration of a peroxide intermediate in our conditions is significant if this reaction is to be practiced on scale. Nonetheless, it is essential to decompose any possible peroxide in any aerobic oxidation before the product is isolated.

### CONCLUSIONS

Conditions to transform azolines to azoles via two efficient and economical aerobic oxidation routes have been developed. These reactions are applicable to a wide range of substrates (electron rich—electron poor), easy to use, involve little waste stream, and are demonstrated on reasonable laboratory scale. Stoichiometric base conditions afford good yields in many cases, but copper-catalyzed conditions afford superior results in most cases. This technology will be useful for building natural products and medicinal entities containing one or more imbedded azole subunits, sensitive labile protons, and electron rich species without the expense of stoichiometric metal oxidants. Further development of aerobic oxidation methods is ongoing in our laboratory.

### EXPERIMENTAL

### General Procedures

All air and water sensitive procedures were carried out either in a Vacuum Atmospheres glove box under nitrogen (2-10 ppm O₂ for all manipulations) or using standard Schlenk techniques under nitrogen. Deuterated NMR solvents were purchased from Cambridge Isotopes Labs and used as received. Other organic solvents and bulk inorganic reagents (e.g. K₂CO₃, NaHCO₃, MgSO₄) were purchased from EM Science and used as received, except where indicated. Iodomethane was purchased from Alfa Aesar and stored, as received, over copper shot. Copper(II) triflate was purchased from Alfa Aesar and used as received. Silica gel (230-400 mesh) was purchased as pre-packed columns from Teledyne.

NMR spectra were recorded on a Varian Mercury 400, 400MR, VNMRS 500, or VNMRS 600 spectrometer. All chemical shifts are reported in units of ppm and referenced to the residual ¹H in the solvent and line-listed according to (s) singlet, (sb) broad singlet, (d) doublet, (t) triplet, (dd) double doublet, etc. ¹³C spectra are delimited by carbon peaks, not carbon count. Melting points were obtained on a mel-temp apparatus and are uncorrected. MALDI mass spectra were obtained on an Applied Biosystems Voyager spectrometer using the evaporated drop method on a coated 96 well plate. The matrix was 2,5-dihydroxybenzoic acid. In a standard preparation, ca. 1 mg analyte and ca. 20 mg matrix were dissolved in a suitable solvent and spotted on the plate with a micro-pipetter. Electrospray ionization (ESI) high-resolution mass spectra were collected at the University of California, Riverside Mass Spectrometry Facility.

### Ligand Screen

Various ligands (table 2) were screened for the oxidation of thiazoline 2 to thiazole 2a. In a representative procedure, the ligand (10 mol %) and Cu(OTf)₂ (10 mol %) were dissolved in N,N-dimethylformamide (DMF) and stirred at room temperature for 30 minutes. Thiazoline 2 (50 mM) and DBU (10 mol %) were added at room temperature. The reaction was stirred at 100° C. in air for 8 hours. Results, as determined by NMR spectroscopy, are summarized in table 2.

### Preparation of Copper Complexes

[(^{Mes}DAB^{Me})Cu^{II}(OH₂)₃]²⁺ 2 Tfo⁻ (1). [^{Mes}DAB^{Me}] ligand (3.00 g, 9.40 mmol) and Cu(OTf)₂ (2.54 g, 7.00 mmol) were dissolved in wet dichloromethane (30 mL) and was allowed to stir at room temperature overnight. The product was precipitated upon addition of hexanes and the crystals were washed with hexanes in air multiple times to yield product as a dark green crystalline solid (1.08 g, 21%). ¹H NMR (400 MHz, CDCl₃): δ=0.88 (sb, 6H), 2.28 (sb, 12H), 2.35 (sb, 6H), 6.90 (sb, 4H). ¹³C NMR cannot be recorded because this compound is paramagnetic. ¹⁹F NMR (376 MHz, CDCl₃): δ=−78.8. MALDI for C₂₄H₃₄CuF₆N₂O₉S₂: calculated [MNa]⁺ 758.08 g/mol, found 758.22, 760.22 g/mol.

In a separate, air- and water free experiment we are able to observe [(^{Mes}DAB^{Me})Cu^{II}(OTf)₂] as a brown crystal. MALDI for C₂₄H₂₈CUF₆N₂O₆S₂: calculated [MNa]⁺ 704.05 g/mol, found 704.19 g/mol.

### Preparation of Azolines

### Methyl 2-Phenyl-4,5-dihydrothiazole-4-carboxylate (2)

Crude 2-phenyl-4,5-dihydrothiazole-4-carboxylic acid (3.5 g, 16.9 mmol) was dissolved in 28 mL DMF at 0° C., to which potassium carbonate (2.57 g, 18.6 mol) was added. After stirring for 30 minutes, iodomethane (2.21 mL, 35.5 mmol) was added and the solution was brought to room temperature and stirred for 1.5 hours until completion by TLC (eluting with 3:1 hexanes:ethyl acetate). The reaction mixture was then diluted in ethyl acetate (40 mL), washed with brine 5 times, and dried over MgSO₄. The crude product mixture was then concentrated under reduced pressure and purified via flash chromatography (5-25% ethyl acetate in hexanes) to yield product as white solid (2.92 g, 13.2 mmol, 23%, 2 steps). Data are consistent with a previously characterized compound.Error! Bookmark not defined. ¹H NMR (400 MHz, CDCl₃): δ=7.87 (m, 2H), 7.47 (m, 1H), 7.41 (m, 2H), 5.29 (t, 1H, J=8.8 Hz), 3.84 (s, 3H), 3.73 (dd, 1H, J₁=11.2 Hz, J₂=8.8 Hz), 3.62 (dd, 1H, J₁=11.2 Hz, J₂=8.8 Hz). All other thiazolines were prepared via a route reported by Kelly et al. (Raman, P; Razavi, H.; Kelly, J. W. Org. Lett. 2000, 2, 3289-3292); the entire disclosure of which is hereby incorporated by reference.

General Procedure for Thiazoline Preparation.Error! Bookmark not defined.

Trityl-protected amide was dissolved in dry dichloromethane (0.05 M solution). Stirring under N₂, a solution of TiCl₄ (1 M in dichloromethane, 3 equiv.) was added and stirred at room temperature overnight until completion. The reaction mixture was then washed with sat. aq. NaHCO₃ twice and dried over MgSO₄. The product was purified via flash chromatography on silica, eluting with ethyl acetate and hexanes.

### Methyl 2-(Naphthalen-2-yl)-4,5-dihydrothiazole-4-carboxylate (5)

5 was prepared from N-(2-napthoyl)-Cys(Trt)-OMe (177 mg, 0.33 mmol) according to the general procedure for thiazoline preparation to give product as oil (23 mg, 26%). ¹H NMR (400 MHz, CDCl₃): δ=8.31 (s, 1H), 8.02 (dd, 1H, J₁=8.0 Hz, J₂=2.0 Hz), 7.91 (dd, 1H, J₁=8.0 Hz, J₂=1.6 Hz), 7.86 (d, 2H, J=8.0 Hz), 7.54 (m, 2H), 5.35 (t, 1 H, J=8.0 Hz), 3.86 (s, 3H), 3.78 (dd, 1H, J₁=12 Hz, J₂=8.0 Hz), 3.69 (dd, 1H, J₁=12 Hz, J₂=8.0 Hz). ¹³C NMR (100 MHz, CDCl₃): δ=171.5, 171.1, 135.0, 132.8, 130.2, 129.8, 129.1, 128.4, 127.9, 127.8, 126.8, 125.0, 78.7, 53.0, 35.6. FT-IR (cm⁻¹): υ=2954, 2929, 1742, 1604. ESI-HRMS for C₁₅H₁₃NO₂S: calculated [MH]⁺ 272.0667 g/mol, found 272.0740 g/mol.

### Methyl 2-(4-Fluorophenyl)thiazole-4-carboxylate (6)

6 was prepared from N-(4-fluorobenzoyl)-Cys(trt)-OMe (750 mg, 1.5 mmol) according to general procedure for thiazoline preparation to give product as white solid (220 mg, 61%), mp 103-105° C. ¹H NMR (500 MHz, CDCl₃): δ=7.87 (ddd, 2H, J₁=8.5 Hz, J₂=5.5 Hz, J₃=2.0 Hz), 7.1 (ddd, 2H, J₁=8.5 Hz, J₂=8 Hz, J₃=2 Hz), 5.28 (t, 1H, J=8.5 Hz), 3.84 (s, 3H), 3.73 (dd, 1H, J₁=11 Hz, J₂=9 Hz), 3.65 (dd, 1H, J₁=11 Hz, J₂=9 Hz). ¹³C NMR (100 MHz, CDCl₃): δ=171.4, 166.3, 163.7, 131.0, 129.1 (d, J_{C-F}=37.2 Hz), 115.8 (d, J_{C-F}=86.8 Hz), 78.6, 53.0, 35.8. FT-IR (cm⁻¹): υ=2953, 1742, 1666, 1603, 1505. ESI-HRMS for C₁₁H₁₀FNO₂S: calculated [MH]⁺ 240.0416 g/mol, found 240.0487.

### Methyl 2-(4-Cyanophenyl)thiazole-4-carboxylate (7)

7 was prepared from N-(2-cyanophenyl)-Cys(trt)-OMe (2.03 g, 4 mmol) according to the general procedure for thiazoline preparation to give product as white solid (151 mg, 15%). Melting Point: 107-108° C. ¹H NMR (500 MHz, CDCl₃): δ=7.96 (dt, 2H, J₁=8.5 Hz, J₂=2.0 Hz), 7.71 (dt, 2H, J₁=9.0 Hz, J₂=2.0 Hz), 5.32 (t, 1H, J=9 Hz), 3.85 (s, 3H), 3.79 (dd, 1H, J₁=11.5 Hz, J₂=9.0 Hz), 3.70 (dd, 1H, J₁=11.3 Hz, J₂=9.0 Hz). ¹³C NMR (100 MHz, CDCl₃): δ=170.9, 147.6, 136.6, 132.4, 129.3, 118.2, 115.2, 78.7, 53.1, 35.9. IR (cm⁻¹): υ=2953, 2920, 2230, 1743. ESI-HRMS for C₁₂H₁₀N₂O₂S: calculated 247.0463 g/mol, found 247.0536 g/mol.

### Methyl 2-(Indol-2-yl)-4,5-dihydrothiazole-4-carboxylate (12)

12 was prepared from methyl 2-(indole-2-carboxamido)-3-(tritylthio)propanoate (200 mg, 0.38 mmol) according to the general procedure for thiazoline preparation to give product as white solid (40 mg, 0.15 mmol, 40%). Melting Point: 141-142° C. ¹H NMR (400 MHz, CDCl₃): δ=9.20 (s, 1H), 7.65 (dd, 1H, J₁=8 Hz, J₂=1.2 Hz), 7.35 (dd, 1H, J₁=8 Hz, J₂=1.2 Hz), 7.29 (ddd, 1H, J₁=8 Hz, J₂=8 Hz, J₃=1.2 Hz), 7.13 (ddd, 1H, J₁=8 Hz, J₂=8 Hz, J₃=1.2 Hz), 6.98 (d, 1H, J=1 Hz), 5.26 (t, 1H, J=8 Hz), 3.84 (s, 3H), 3.76 (dd, 1H, J₁=12 Hz, J₂=8 Hz), 3.68 (dd, 1H, J₁=12, Hz, J₂=8 Hz). ¹³C NMR (100 MHz, CDCl₃): δ=171.3, 163.0, 137.1, 130.2, 127.9, 152.2, 122.1, 120.2, 111.7, 108.7, 77.7, 53.0, 35.6. FT-IR (cm⁻¹): υ=3061, 2951, 1739, 1603, 1518. ESI-HRMS for C₁₃H₁₂N₂O₂S: calculated [MH]⁺ 261.0619 g/mol, found 261.0691 g/mol.

### Methyl 2-(1-methylindol-2-yl)-4,5-dihydrothiazole-4-carboxylate (13)

13 was prepared from methyl 2-(1-methylindole-2-carboxamido)-3-(tritylthio)propanoate (1.1 g, 2.1 mmol) according to the general procedure for thiazoline preparation to give product as white solid (120 mg, 0.44 mmol, 21%). Melting Point: 78-80° C. ¹H NMR (400 MHz, CDCl₃): δ=7.64 (dt, 1H, J₁=8 Hz, J₂=1.2 Hz), 7.37 (d, 1H, J=8 Hz), 7.33 (ddd, 1H, J₁=8 Hz, J₂=8 Hz, J₃=1.2 Hz), 7.14 (ddd, 1H, J₁=8 Hz, J₂=8 Hz, J₃=1.2 Hz), 7.16 (s, 1H), 5.37 (dd, 1H, J₁=8 Hz, J₂=8 Hz), 4.12 (s, 3H), 3.84 (s, 3H), 3.65 (dd, 1H, J₁=8 Hz, J₂=12 Hz), 3.59 (dd, 1H, J₁=8 Hz, J₂=12 Hz). ¹³C NMR (100 MHz, CDCl₃): δ=171.6, 163.3, 139.9, 131.0, 126.7, 124.7, 122.0, 120.5, 110.3, 110.2, 79.1, 52.9, 34.8, 32.3. FT-IR (cm⁻¹): υ=3060, 2951, 1741, 1660, 1603, 1510. ESI-HRMS for C₁₄H₁₄N₂O₂S: calculated [MH]⁺ 275.0776 g/mol, found 275.0850 g/mol.

### General Procedure for Catalytic Oxidation.

Azoline was dissolved in DMF at room temperature (50 mM). After the addition of (DAB)Cu^{II} complex 1 (10 mol %) and DBU (10 mol %, or other if specified), the reaction was allowed to stir at 100° C. in air until complete by TLC (eluting with 3:1 hexanes:ethyl acetate). The solution was then diluted with ethyl acetate, washed with deionized water, and dried over MgSO₄. The crude product was purified via flash chromatography on silica, eluting with 0-20% ethyl acetate in hexanes, to give the corresponding azole.

### General Procedure for Base-Promoted Oxidation of Azolines.

Azoline was dissolved in DMF at room temperature (50 mM). After the addition of DBU (1.1 equiv., or other if specified), the reaction was allowed to stirring at 70° C. in air until complete by TLC (eluting with 3:1 hexanes:ethyl acetate). The solution was then diluted with ethyl acetate, washed with deionized water and dried over MgSO₄. The crude product was purified via flash chromatography on silica, eluting with 0-20% ethyl acetate in hexanes, to give the corresponding azole.

### Methyl 2-Phenylthiazole-4-carboxylate (2a)

2a was prepared from methyl 2-phenyl-4,5-dihydrothiazole-4-carboxylate (22 mg, 0.1 mmol) according to the catalytic procedure (8 hours, 19 mg, 87%) or base-promoted procedure (0.5 hours, 15 mg, 66%) to give 2a as white solid. Data are consistent with a previously characterized compound. (Gududuru, V.; Hurh, E.; Dalton, J. T.; Miller, D. D. J. Med. Chem. 2005, 48, 2584-2588). ¹H NMR (400 MHz, CDCl₃): δ=8.37 (s, 1H), 7.98 (m, 2H), 7.47 (m, 3H), 3.91 (s, 3H).

### Methyl 2-(4-Nitrophenyl)thiazole-4-carboxylate (3a)

3a was prepared from methyl 2-(4-nitrophenyl)-4,5-dihydrothiazole-4-carboxylate (53 mg, 0.2 mmol)Error! Bookmark not defined. according to the general catalytic procedure (3 hours, 41 mg, 78%) or a variant of the base-promoted procedure wherein only 10 mol % of DBU is incorporated (1 hour, 36 mg, 69%). Melting Point: 224-227° C. ¹H NMR (400 MHz, CDCl₃): δ=8.31 (d, 2H, J=8 Hz), 8.29 (s, 1H), 8.18 (d, 2H, J=8 Hz), 3.98 (s, 3 H). ¹³C NMR (100 MHz, CDCl₃): δ=161.8, 148.8, 138.3, 129.7, 129.0, 127.9, 124.6, 52.9, 29.9. IR (cm⁻¹): υ=3125, 3092, 1721. ESI-HRMS for C₁₁H₈N₂O₄S: calculated [MH]⁺ 265.0205 g/mol, found: 265.0278 g/mol.

### Methyl 2-(4-Methoxyphenyl)thiazole-4-carboxylate (4a)

4a was prepared from methyl 2-(4-methoxyphenyl)-4,5-dihydrothiazole-4-carboxylate (25 mg, 0.1 mmol)Error! Bookmark not defined. according to the general catalytic procedure (8 hours, 17 mg, 68%) or base-promoted procedure (4 hours, 14 mg, 58%). Melting Point: 67-79° C. ¹H NMR (400 MHz, CDCl₃): δ=8.10 (s, 1H), 7.96 (d, 2H, J=8 Hz), 6.97 (d, 2H, J=8 Hz), 3.97 (s, 3H), 3.87 (s, 3H). ¹³C NMR (100 MHz, CDCl₃): δ=169.0, 162.2, 161.8, 147.6, 128.7, 126.7, 125.8, 114.4, 55.6, 52.6. FT-IR (cm⁻¹): υ=3119, 3025, 1740, 1710. ESI-HRMS for C₁₂H₁₁NO₃S: calculated 250.0460 g/mol, found 250.0532 g/mol.

### Methyl 2-(Napthalen-2-yl)thiazole-4-carboxylate (5a)

5a was prepared from methyl 2-(naphthalen-2-yl)-4,5-dihydrothiazole-4-carboxylate (5, 20 mg, 0.74 mmol) according to the catalytic procedure (8.5 hours, 16 mg, 79%) or base-promoted procedure (1 hour, 15 mg, 77%) to give 5a. ¹H NMR (400 MHz, CDCl₃): δ=8.52 (s, 1H), 8.22 (s, 1H), 8.09 (d, 1H, J=8 Hz), 7.93 (m, 2H), 7.85 (m, 1H), 7.54 (m, 2 H), 4.01 (s, 3H). ¹³C NMR (100 MHz, CDCl₃): δ=169.3, 162.2, 148.1, 134.6, 133.3, 130.3, 129.1, 129.0, 128.1, 127.6, 127.2, 126.9, 124.3, 52.8, 29.9. FT-IR (cm¹): υ=3138, 3048, 1733. ESI-HRMS for C₁₅H₁₁NO₂S: calculated [MH]⁺ 270.0510 g/mol, found 270.0583 g/mol.

### Methyl 2-(4-Fluorophenyl)thiazole-4-carboxylate (6a)

6a was prepared from methyl 2-(4-fluorophenyl)-4,5-dihydrothiazole-4-carboxylate (6, 20 mg, 0.084 mmol) according to the catalytic procedure (2 hours, 12 mg, 58%) or base-promoted oxidation (45 minutes, 9 mg, 44%) to give 6a. ¹H NMR (400 MHz, CDCl₃): δ=8.16 (s, 1H), 8.00 (m, 2H), 7.15 (t, 2H, J=8.4 Hz), 3.98 (s, 3H). ¹³C NMR (100 MHz CDCl₃) δ: 165.7, 163.2, 162.1, 147.9, 129.30, 129.1 (d, J_{C-F}=33.6 Hz), 127.5, 116.3 (d, J_{C-F}=88.4 Hz), 52.7. FT-IR (cm⁻¹): n=3133, 3108, 1750. ESI-HRMS for C₁₁H₈FNO₂S: calculated [MH]⁺ 238.0260 g/mol, found 238.0333 g/mol.

### Methyl 2-(4-Cyanophenyl)thiazole-4-carboxylate (7a)

7a was prepared from methyl 2-(4-cyanophenyl)-4,5-dihydrothiazole-4-carboxylate (7, 20 mg, 0.81 mmol) according to the catalytic procedure (4 hours, 14 mg, 69%) or base-promoted procedure (45 minutes, 9 mg, 44%) to give 7a. Melting Point: 199-201° C. ¹H NMR (500 MHz, CDCl₃): δ=8.27 (s, 1H), 8.13 (dd, 2H, J₁=8.5 Hz, J₂=2.5 Hz), 7.76 (dd, 2H, J₁=8.5 Hz, J₂=2.5 Hz), 4.0 (s, 3H). ¹³C NMR (100 MHz, CDCl₃): δ=166.6, 161.8, 148.6, 136.7, 133.0, 128.7, 127.6, 118.4, 114.3, 52.9. FT-IR (cm⁻¹): υ=3133, 2233, 1747. ESI-HRMS for C₁₂H₈N₂O₂S: calculated [MH]⁺ 245.0306 g/mol, found 245.0379 g/mol.

### Methyl 2-Phenyloxazole-4-carboxylate (8a)

8a was prepared from methyl 2-phenyl-4,5-dihydrooxazole-4-carboxylate (20 mg, 0.1 mmol)Error! Bookmark not defined. according to a variant of the catalytic procedure wherein 30 mol % of base is added (9 hours, 4 mg, 18%) or base-promoted procedure (6 hours, 3 mg, 16%). Data are consistent with a previously characterized compound. (Shapiro, R. J. Org. Chem. 1993, 58, 5759-5764). ¹H NMR (400 MHz, CDCl₃): δ=8.31 (s, 1H), 8.13 (d, 2H, J=8 Hz), 7.49 (m, 3H), 3.97 (s, 3H).

### Methyl 2-(4-Nitrophenyl)oxazole-4-carboxylate (9a)

9a was prepared from methyl 2-(4-nitrophenyl)-4,5-dihydrooxazole-4-carboxylate (20 mg 0.08 mmol) (Castellano, S.; Kuck, D.; Sala, M.; Novellino, E.; Lyko, F.; Sbardella, G. J. Med. Chem. 2008, 51, 2321-2325. (b) Phillips, A. J.; Uto, Y.; Wipf, P.; Reno, M. J.; Williams, D. R. Org. Lett. 2000, 2, 1165-1168) according to a variant of the catalytic procedure wherein 30 mol % of base is added (12 hours, 7 mg, 37%) or base-promoted procedure (2 hours, 8 mg, 41%). Data are consistent with a previously characterized compound. (Tsuyoshi, S.; Hiroshi, T.; Kagoshima, H.; Yamamoto Y.; Hosokawa, T.; Toshiyuhi, K.; Nobuhisa, M.; Takuya, U.; Issei, A.; Junichi, K.; Tetsunori, F.; Aki, Y.; Tetsuji, N. PCT Int. Appl. (2009)). ¹H NMR (400 MHz, CDCl₃): δ=8.38 (s, 1H), 8.36 (dt, 2H, J₁=8 Hz, J₂=2.4 Hz), 8.31 (dt, 2H, J₁=8 Hz, J₂=2.4 Hz), 3.99 (s, 3H).

### Methyl 2-Methylthiazole-4-carboxylate (10a)

10a was prepared from methyl 2-methyl-4,5-dihydrothiazole-4-carboxylate (20 mg, 0.12 mmol) (Emtenas, H.; Alderin, L.; Almqvist, F. J. Org. Chem. 2001, 66, 6756-6761.) according to the catalytic procedure (8 hours, 5 mg, 24%) or base-promoted procedure (5 hours, 8 mg, 39%). Data are consistent with a previously characterized compound. (Evans, D. L.; Minster, D. K.; Jordis, U.; Hecht, S. M.; Mazzu Jr., A. L.; Meyers, A. I. J. Org. Chem. 1979, 44, 497-501.) ¹H NMR (400 MHz, CDCl₃): δ=8.05 (s, 1H), 3.95 (s, 3H), 2.77 (s, 3 H).

### Methyl 2-Phenethylthiazole-4-carboxylate (11a)

11a was prepared from methyl 2-phenethyl-4,5-dihydrothiazole-4-carboxylate (11, 20 mg, 0.08 mmol)Error! Bookmark not defined. according to the catalytic procedure (12 hours, 9 mg, 45%) or base-promoted procedure (6 hours, 10 mg, 51%). ¹H NMR (400 MHz, CDCl₃): δ=8.05 (s 1H), 7.3 (m, 2H), 7.21 (m, 3H), 3.96 (s, 3H), 3.38 (t, 2H, J=8 Hz), 3.18 (t, 2H, J=8 Hz). ¹³C NMR (100 MHz, CDCl₃): δ=171.1, 162.1, 146.6, 140.0, 128.8, 128.6, 127.4, 126.7, 52.6, 36.1, 35.4. FT-IR (cm⁻¹): υ=3119, 2954, 1721. ESI-HRMS for C₁₃H₁₃NO₂S: calculated [W]⁺ 248.0667 g/mol, found 248.0740 g/mol.

### Methyl 2-(Indol-2-yl)thiazole-4-carboxylate (12a)

12a was prepared from methyl 2-(indol-2-yl)-4,5-dihydrothiazole-4-carboxylate (20 mg, 0.077 mmol) according to the catalytic procedure (6 hours, 11 mg, 55%). Melting Point: 69-71° C. ¹H NMR (400 MHz, CDCl₃): δ=9.33 (s, 1H), 8.13 (s 1H), 7.65 (dd, 1H J₁=8 Hz, J₂=0.8 Hz), 7.40 (dd, 1H J₁=8 Hz, J₂=0.8 Hz), 7.28 (ddd, 1H, J₁=8 Hz, J₁=8 Hz, J₃=0.8 Hz), 7.15 (ddd, 1H, J₁=8 Hz, J₂=8 Hz, J₃=0.8 Hz), 7.05 (dd, 1H, J₁=2 Hz, J₂=0.8 Hz), 3.99 (s, 3H). ¹³C NMR (100 MHz, CDCl₃): δ=161.8, 161.1, 147.2, 136.8, 130.6, 128.4, 126.8, 124.7, 121.6, 121.0, 111.6, 104.3, 52.7. FT-IR (cm⁻¹): υ=2921, 2852, 1732, 1717. ESI-HRMS for C₁₃H₁₀N₂O₂S: calculated [MH]⁺ 259.0463 g/mol, found 259.0536 g/mol.

### Methyl 2-(1-Methyl-indol-2-yl)thiazole-4-carboxylate (13a)

13a was prepared from methyl 2-(1-methylindol-2-yl)-4,5-dihydrothiazole-4-carboxylate (20 mg, 0.073 mmol) according to the catalytic procedure (14 hours, 13 mg, 65%) or base-promoted procedure (30 minutes, 9 mg, 45%). Melting Point: 124-127° C. ¹H NMR (400 MHz, CDCl₃): δ=8.15 (s, 1H), 7.64 (d, 1H, J=8 Hz), 7.40 (d, 1H, J=8.8 Hz), 7.32 (ddd, 1H, J₁=8 Hz, J₂=8 Hz, J₃=1.2 Hz), 7.16 (ddd, 1H, J₁=8 Hz, J₂=8 Hz, J₃=1.2 Hz), 7.04 (s, 1H), 4.21 (s, 3H), 3.98 (s, 3H). ¹³C NMR (100 MHz, CDCl₃): δ=162.0, 161.6, 147.6, 139.5, 131.6, 127.2, 127.1, 124.1, 121.5, 120.7, 110.3, 106.1, 52.6, 32.1. FT-IR (cm⁻¹): υ=2953, 2925, 1732, 1552. ESI-HRMS for C₁₄H₁₂N₂O₂S: calculated [MH]⁺ 273.0619 g/mol, found 273.0697 g/mol.

### Methyl 4-hydroxy-2-phenyl-4,5-dihydrothiazole-4-carboxylate (15)

15 was prepared from methyl 2-phenyl-4,5-dihydrothiazole-4-carboxylate via the general procedure for base-promoted oxidation in which the reaction was stopped after 15 minutes. ¹H NMR: 7.89 (dd, 2H, J=8 Hz, J=1.2 Hz), 7.51 (tt, 1H, J=8 Hz, J=8 Hz), 7.42 (tt, 2H, J=8 hz, J=1.2 Hz), 4.18 (s, 1H), 4.02 (dd, 2H, J=12 Hz, J=1.2 Hz), 3.89 (s, 3H), 3.55 (d, 1H, J=12 Hz). MALDI for C₁₁H₁₁NO₃S: Calculated [MH]⁺ 238.04 g/mol, found 238.00 g/mol.

### Scale Up Reaction

In a 3-neck round bottom flask, N,N′-(butane-2,3-diylidene)bis(2,4,6-trimethylaniline) (145 mg, 0.45 mmol)Error! Bookmark not defined. and copper(II) triflate (164 mg, 0.45 mmol) were stirred in DMF at room temperature for 30 minutes. DBU (0.068 mL, 0.45 mmol) and methyl 2-phenyl-4,5,-dihydrothiazole-4-carboxylate (1.0 g, 4.5 mmol) were added sequentially. A condenser was then attached to the flask, which was then placed in a 100° C. oil bath. A gentle stream of compressed air was bubbled into the reaction, which was stirred for 18 hours. The reaction mixture was diluted with ethyl acetate and washed with deionized water three times then dried over MgSO₄. The crude reaction mixture was then concentrated under reduced pressure and purified via column chromatography (5-25% hexanes in ethyl acetate) to yield desired product (791 mg, 3.6 mmol, 80%).

### Oxidation of Thiazoline 2 in the Presence of H₂¹⁸O

Thiazoline was dissolved in DMF (previously dried over CaH) at room temperature (50 mM). H₂¹⁸O (1.2 equiv.) and DBU (1.1 equiv.) were added and the reaction was stirred at 70° C. in air for 30 minutes. An aliquot of the reaction mixture was analyzed by MALDI and compared to an isolated sample of angular hydroxide thiazoline 15 made as a reaction intermediate by the general procedure for base-promoted oxidation. Vanishingly little additional incorporation of ¹⁸O was observed (See Supporting Information of a graphical MALDI spectrum).

Oxidation of 2 with K₂CO₃

Reaction of thiazoline 2 with K₂CO₃ (1 equiv.) and catalyst 1 in DMF (2 mL) produced thiazole 2a in 30% yield as well as a mixture of angular hydroxide 15 and an unknown intermediate, which is purportedly an angular peroxide, in a ratio of ca. 1:1.3 ratio, 22% and ca. 26% isolated yields respectively.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method of forming an azole, the method comprising:
a) reacting a compound having formula (I) with a copper-containing catalyst in the presence of a base or proton acceptor to form a compound having formula (II): 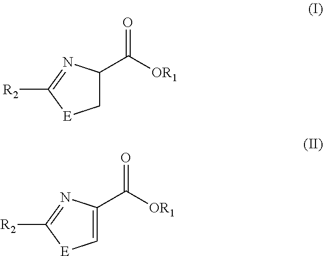
wherein:
R₁ is C₁-C₁₀ alkyl;
R₂ is an optionally substituted phenyl, optionally substituted C₅-C₁₈ aryl, or optionally substituted C₅-C₁₈ heteroaryl; and
E is O, S, or N.

2. The method of claim 1 wherein R₁ is methyl, ethyl, butyl or pentyl and E is O or S.

3. The method of claim 1 wherein the copper-containing catalyst has the following formula: 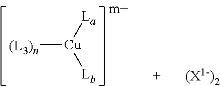
wherein:
Lₐ, L_{b}, and L₃ are each independently two electron ligands;
X¹⁻ is a negatively charged counter ion;
Cu is in Cu(I) or Cu(II)
n is 0, 1, 2, or 3; and
m is 0, 1, or 2.

4. The method of claim 3 wherein L₃ is a neutral ligand.

5. The method of claim 4 wherein L₃ is H₂O, NH₃, C₁₋₅ primary amine, C₂₋₆ secondary amine, C₃₋₉ tertiary amine, PH₃, C₁₋₅ primary phosphines, C₂₋₆ secondary phosphine, C₃₋₉ tertilry phosphines, C₁₋₅ alcohols, CO, N₂, C₂₋₈ alkenes, or C₂₋₈ alkynes.

6. The method of claim 3 wherein L₃ is halide, CF₃SO₃⁻, C₁₋₅ alkoxide, or C₁₋₅ carboxylate.

7. The method of claim 1 wherein the copper-containing catalyst has the following formula: 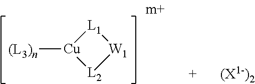
wherein:
Lₐ, L_{b}, and L₃ are each independently two electron ligands;
n is from 0, 1, 2, or 3;
W₁ is an absent or a C₁₋₁₈ hydrocarbon moiety attached to L₁ and L₂
X¹⁻ is a negatively charged counter ion;
Cu is in Cu(I) or Cu(II)
n is 0, 1, 2, or 3; and
m is 0, 1, or 2.

9. The method of claim 8 wherein X⁻ is halide. CF₃SO₃⁻, C₁₋₅ alkoxide, or C₁₋₅ carboxylate.

10. The method of claim 8 wherein L₁-W-L₂ is selected from the group consisting of:

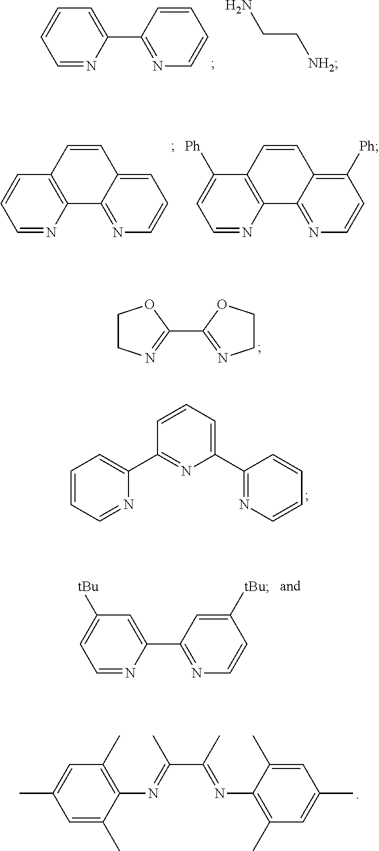

11. The method of claim 1 wherein the compound having formula (I) is selected from the group consisting of compounds having formula (V) and (VI):

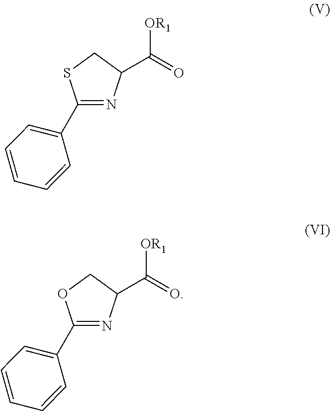

12. The method of claim 11 wherein the phenyl group in compounds (V) or (VI) is substituted with C₁₋₆ alkyl, fluorine, chlorine, bromine, cyano, or nitro.

13. The method of claim 1 wherein compound (I) is selected from the group consisting of:

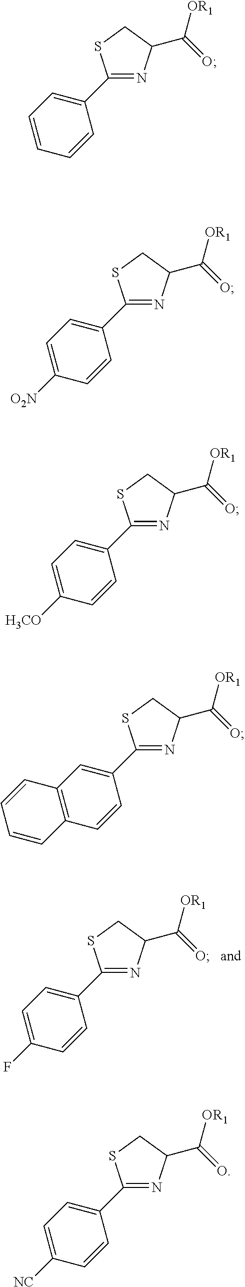

14. The method of claim 1 wherein compound (I) is selected from the group consisting of:

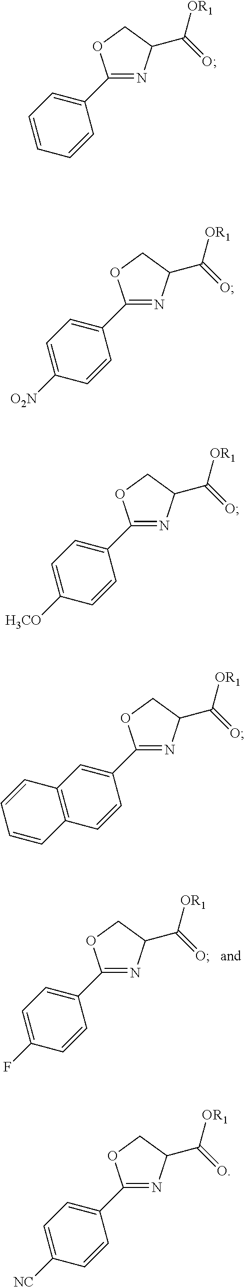

15. The method of claim 1 wherein R₂ is: 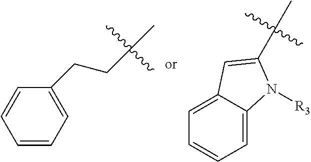
and R₃ is hydrogen or C₁₋₁₀ alkyl.

16. The method of claim 1 wherein the reaction of step a) is performed in the presence of molecular oxygen.

17. The method of claim 1 wherein the base or proton acceptor is 1,8-diazabicyclo [5.4.0]undec-7-ene, 1,8-Bis(dimethylamino)naphthalene (proton Sponge™), 1,8-bis(hexamethyltriaminophosphazenyl)naphthalene, diisopropyl ethyl amine, potassium tert-butoxide, or potassium carbonate.

18. A method of forming an azole, the method comprising:
a) reacting a compound having formula (I) with a base or proton acceptor in the presence of molecular oxygen to form a compound having formula (II): 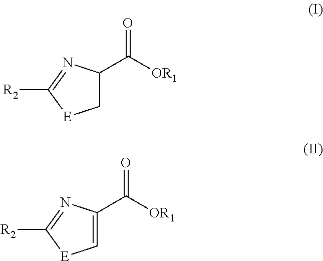
wherein:
R₁ is C₁-C₁₀ alkyl;
R₂ is an optionally substituted phenyl, optionally substituted C₅-C₁₈ aryl, or optionally substituted C₅-C₁₈ heteroaryl; and
E is O, S, or N,

19. The method of claim 18 wherein R₂ is methyl, ethyl, butyl or pentyl and E is O or S.

20. The method of claim 18 wherein the compound having formula (I) is selected from the group consisting of compounds having formula (V) and (VI):

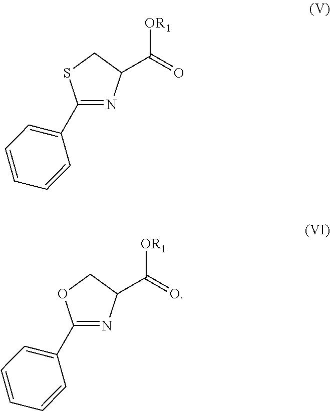

21. The method of claim 20 wherein the phenyl group in compounds (V) or (VI) is substituted with C₁₋₆ alkyl, fluorine, chlorine, bromine, cyano, or nitro.

22. The method of claim 18 wherein compound (I) is selected from the group consisting of:

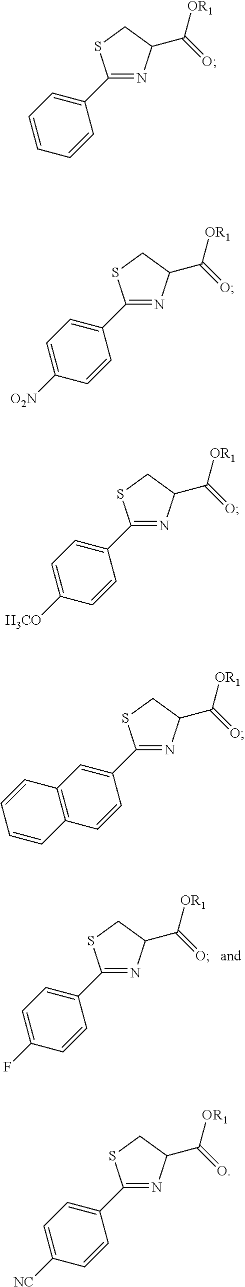

23. The method of claim 18 wherein compound (I) is selected from the group consisting of:

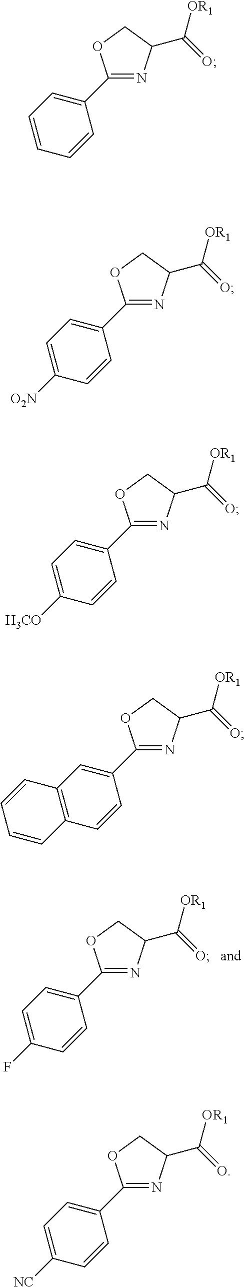

24. The method of claim 18 wherein R₂ is: 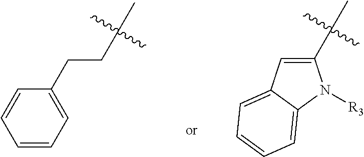
and R₃ is hydrogen or C₁₋₁₀ alkyl.

25. The method of claim 18 wherein the reaction of step a) is performed in the presence of molecular oxygen.

26. The method of claim 18 wherein the base or proton acceptor is 1,8-diazabicyclo[5.4.0]undec-7-ene.
